(19) United States
(12) Patent Application Publication
(10) Pub. No.: US 2021/0393902 A1
(43) Pub. Date: Dec. 23, 2021

(54) ONE-TOUCH VENTILATION MODE

(71) Applicant: Covidien LP, Mansfield, MA (US)

(72) Inventors: Nancy F. Dong, San Marcos, CA (US); Gabriel Sanchez, Valley Center, CA (US); Kun Li, San Diego, CA (US)

(73) Assignee: Covidien LP, Mansfield, MA (US)

(21) Appl. No.: 17/354,049

(22) Filed: Jun. 22, 2021

Related U.S. Application Data
(60) Provisional application No. 63/042,737, filed on Jun. 23, 2020.

(51) Int. Cl. A61M 16/00

(52) U.S. Cl. CPC A61M 16/022; A61M 2205/505; A61M 16/0051; A61M 16/0003

(57) ABSTRACT

Systems and methods for one-touch ventilation mode are disclosed. In examples, settings for a medical ventilator are determined and delivered to a patient with a minimum of one input parameter. The one-touch ventilation mode may reference or apply one or more respiratory mechanics planes to determine desired ventilation parameters. In an example, the input parameter may be mapped to initial ventilation settings on a respiratory mechanics plane. During ventilation delivered according to the initial ventilation settings, ventilation data may be obtained. Based on the ventilation data, one or more ventilation strategies may be implemented, including breath type strategy, alarming strategy, triggering/cycling strategy, and PEEP strategy. Updated ventilation settings may be determined based on the ventilation data and/or the ventilation strategy.

### CROSS-REFERENCE TO RELATED APPLICATIONS

This application claims the benefit of U.S. Provisional Application No. 63/042,737, filed Jun. 23, 2020, the complete disclosure of which is hereby incorporated herein by reference in its entirety.

### INTRODUCTION

Medical ventilator systems have long been used to provide ventilatory and supplemental oxygen support to patients. These ventilators typically comprise a connection for pressurized gas (air, oxygen) that is delivered to the patient through a conduit or tubing. As each patient may require a different ventilation strategy, modern ventilators may be customized for the particular needs of an individual patient. For example, several different ventilator modes or settings have been created to provide better ventilation for patients in different scenarios, such as mandatory ventilation modes, spontaneous ventilation modes, and assist-control ventilation modes. Ventilators monitor a variety of patient parameters and are well equipped to provide reports and other information regarding a patient's condition.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Among other things, aspects of the present disclosure include systems and methods for one-touch ventilation. More specifically, this disclosure describes systems and methods for providing support to patients through a variety of algorithms and strategies with a minimum of one input parameter. The one-touch ventilation may be a mode of a ventilator. The one-touch ventilation mode may reference one or more respiratory mechanics planes to determine desired ventilation parameters. A variety of algorithms may then be implemented based on the respiratory mechanics planes, such as volume-targeted pressure control and lung conditions identification component. Additionally, the one-touch ventilation mode may determine a variety of strategies for ventilating the patient, including breath type strategy, alarming strategy, triggering/cycling strategy, and PEEP strategy.

In an aspect, a method for controlling a medical ventilator is disclosed. The method includes receiving, at the medical ventilator, an input of intrinsic information associated with a patient and applying the intrinsic information to a respiratory mechanics plane to generate initial ventilation settings. The method further includes delivering pressurized ventilation according to the initial ventilation settings and acquiring ventilation data. Further, the method includes applying the acquired ventilation data to the respiratory mechanics plane to generate updated ventilation settings and delivering subsequent ventilation according to the updated ventilation settings.

In an example, the respiratory mechanics plane is at least one of: a normalized respiratory mechanics (NRM) plane and a respiratory rate (RR) plane. In another example, the acquired ventilation data is a compliance of the patient and the updated ventilation settings are associated with a desired distending pressure. In a further example, applying the acquired ventilation data to the respiratory mechanics plane includes determining a patient status point on the respiratory mechanics plane. In yet another example, the respiratory mechanics plane includes a preferred region of ventilation, and wherein applying the acquired ventilation data to the respiratory mechanics plane further includes comparing the patient status point and the preferred region of ventilation. In still a further example, the intrinsic information is a predicted body weight of the patient. In another example, the acquired ventilation data is one of: a spontaneous breath rate, an expiratory time constant, PEEP level, a patient effort, an airway pressure, a compliance, and an oxygen saturation. In a further example, the acquired ventilation data is associated with a ventilation strategy, wherein the ventilation strategy is at least one of: a breath type strategy, an alarming strategy, a triggering strategy, a cycling strategy, and a PEEP strategy. In yet another example, the acquired ventilation data is the expiratory time constant and the ventilation strategy is the PEEP strategy. In still a further example, delivering subsequent ventilation includes changing one of: an inhalation flow or an exhalation pressure.

In another aspect, a method for controlling a medical ventilator is disclosed. The method includes receiving an input of intrinsic information associated with a patient and mapping the intrinsic information to initial ventilation settings on a respiratory mechanics plane, the initial ventilation settings including at least an initial tidal volume setting and an initial pressure setting. The method further includes delivering initial ventilation according to the initial ventilation settings. During initial ventilation, the method includes determining a net flow value. Based on the net flow value, the method further includes determining a lung condition. Based on the lung condition, the method further includes determining a trigger type and a PEEP protocol. Based on the determined trigger type and PEEP protocol, the method includes delivering subsequent ventilation.

In an example, the method further includes increasing the PEEP level, based on the PEEP protocol. In another example, the method further includes applying the PEEP protocol to the respiratory mechanics plane to generate updated ventilation settings; and delivering the updated ventilation settings. In yet another example, determining the lung condition includes: determining an expiratory time constant of an exhalation phase of the patient; and comparing the expiratory time constant with a time constant threshold to identify the lung condition. In still a further example, the trigger type is one of: a flow trigger type, a pressure trigger type, a signal distortion trigger type, or a synchronized trigger type.

In a further aspect, a method for controlling a medical ventilator is disclosed. The method includes initiating positive pressure ventilation with one-touch input, the one-touch input indicating intrinsic information associated with the patient. The method further includes mapping the intrinsic information on a respiratory mechanics plane to determine initial ventilation settings and delivering the positive pressure ventilation according to the initial ventilation settings. During ventilation of the patient, the method includes measuring ventilation data including at least one of: a net flow value, an airway pressure value, or a spontaneous respiratory rate value. The method further includes mapping the measured ventilation data on the respiratory mechanics plane to determine updated ventilation settings; and delivering subsequent positive pressure ventilation according to the updated ventilation settings without requiring further input from a clinician.

In an example, the initial ventilation settings include at least an initial tidal volume setting and an initial pressure setting. In another example, the measured ventilation data includes the net flow value, the airway pressure value, and the spontaneous respiratory rate value. In a further example, the measured ventilation data includes a lung condition determined based on the net flow value. In yet another example, the measured ventilation data includes a lung condition determined based on the net flow value and a patient tidal volume based on the airway pressure value.

It is to be understood that both the foregoing general description and the following Detailed Description are explanatory and are intended to provide further aspects and examples of the disclosure as claimed.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 is a diagram illustrating an example of a ventilator connected to a human patient.

FIG. 2 is a block-diagram illustrating an example of a ventilator system.

FIG. 3A is a chart illustrating an example of a normalized respiratory mechanics (NRM) plane.

FIG. 3B is a chart illustrating a normalized respiratory mechanics (NRM) plane with provided patient temporal status points.

FIG. 3C is a chart illustrating a respiratory rate (RR) plane, that shows a relationship between an input parameter and respiratory rate.

FIG. 4A is block diagram illustrating a schematic flowchart for one-touch ventilation mode.

FIG. 4B is a block diagram illustrating a volume targeted pressure control system, shown as a subset of the schematic flowchart of one-touch ventilation mode shown in FIG. 4A.

FIG. 5 is a flowchart illustrating a method for one-touch ventilation mode.

FIG. 6 is a flowchart illustrating a method for one-touch ventilation mode, including ventilation strategies.

While examples of the disclosure are amenable to various modifications and alternative forms, specific aspects have been shown by way of example in the drawings and are described in detail below. The intention is not to limit the scope of the disclosure to the particular aspects described. On the contrary, the disclosure is intended to cover all modifications, equivalents, and alternatives falling within the scope of the disclosure and the appended claims.

### DETAILED DESCRIPTION

As discussed briefly above, medical ventilators are used to provide breathing gases to patients who are otherwise unable to breathe sufficiently. In modern medical facilities, pressurized air and oxygen sources are often available from wall outlets, tanks, or other sources of pressurized gases. Accordingly, ventilators may provide pressure regulating valves (or regulators) connected to centralized sources of pressurized air and pressurized oxygen. The regulating valves function to regulate flow so that respiratory gases having a desired concentration are supplied to the patient at desired pressures and flow rates. Further, as each patient may require a different ventilation strategy, modern ventilators may be customized for the particular needs of an individual patient.

For the purposes of this disclosure, a “breath” refers to a single cycle of inspiration and exhalation delivered with the assistance of a ventilator. The term “breath type” refers to some specific definition or set of rules dictating how the pressure and flow of respiratory gas is controlled by the ventilator during a breath.

A ventilation “mode,” on the other hand, is a set of rules controlling how multiple subsequent breaths should be delivered. Modes may be mandatory, as controlled by the ventilator, or spontaneous, that allows a breath to be delivered or controlled upon detection of a patient's effort to inhale, exhale or both. For example, a simple mandatory mode of ventilation is to deliver one breath of a specified mandatory breath type at a clinician-selected respiratory rate, f (e.g., one breath every 6 seconds). Typically, ventilators will continue to provide breaths of the specified breath type as dictated by the rules defining the mode, until the mode is changed by a clinician. For example, breath types may be mandatory mode breath types where the initiation and termination of the breath is made by the ventilator, or spontaneous mode breath types where the breath is initiated and terminated by the patient. Examples of breath types utilized in the spontaneous mode of ventilation include proportional assist (PA) breath type, volume support (VS) breath type, pressure support (PS) breath type, etc. Examples of mandatory breath types include a volume control breath type, a pressure control breath type, volume-targeted pressure control breath type etc.

In recent years, there has been a dizzying proliferation of medical ventilation modes, driven by technological advances and market pressures. As an example, the first respiratory care equipment books published in the United States named three modes (i.e., control, assist, and assist/control). More recent editions of respiratory care equipment books list upwards of 174 unique names for modes of medical ventilation. The large quantity of available modes may sometimes cause clinician confusion, frustration, and/or waste of time. Moreover, each ventilation mode provides multiple settings that require clinicians to have a good understanding of the patient's disease conditions and recovery progress in order to achieve patient-specific support. Thus, selection of a proper ventilation mode requires clinician time and effort, even for initial ventilation settings.

Because a proper selection of a ventilation mode, and the settings therefore, is time-intensive and requires substantial knowledge, patients may not have optimized care under conditions where clinicians lack time or experience. Time-sparse conditions may include a small ratio of clinicians to patients, such as when patient influx is high and/or when available clinicians are limited. As another example, a lack of clinician time may occur in situations such as epidemics, economic crisis, limited hospital funding or resources, wartime, etc.

Among other things, the systems and methods disclosed herein address these circumstances by providing a one-touch ventilation mode. The one-touch ventilation may require only a simple input from the clinician to provide ventilatory support to a patient, thus resulting in efficient clinician time and reducing clinical errors in mode selection. In an example, the simple input may include a confirmation (e.g., selection of a control associated with confirm, implement, initiate, etc.). For example, one-touch ventilation mode may receive an input parameter and a confirmation. Alternatively, one-touch ventilation mode may automatically initiate without additional inputs. Additionally, the ventilator may utilize a normalized adult respiratory mechanics (NRM) plane that identifies a preferred region of ventilation on a graph of normalized tidal volume (V_{T}) versus distending pressure (P_{dist}) that not only shows preferred ventilation regions for the simple input by the clinician, but also helps reduce the possibility for lung injuries. Aspects of the described NRM plane are described in U.S. Publication No. 2019/0143058 and U.S. Publication No. 2019/0143059, which are each incorporated by reference in their entireties. With these concepts in mind, several examples of one-touch ventilation mode methods and systems are discussed below.

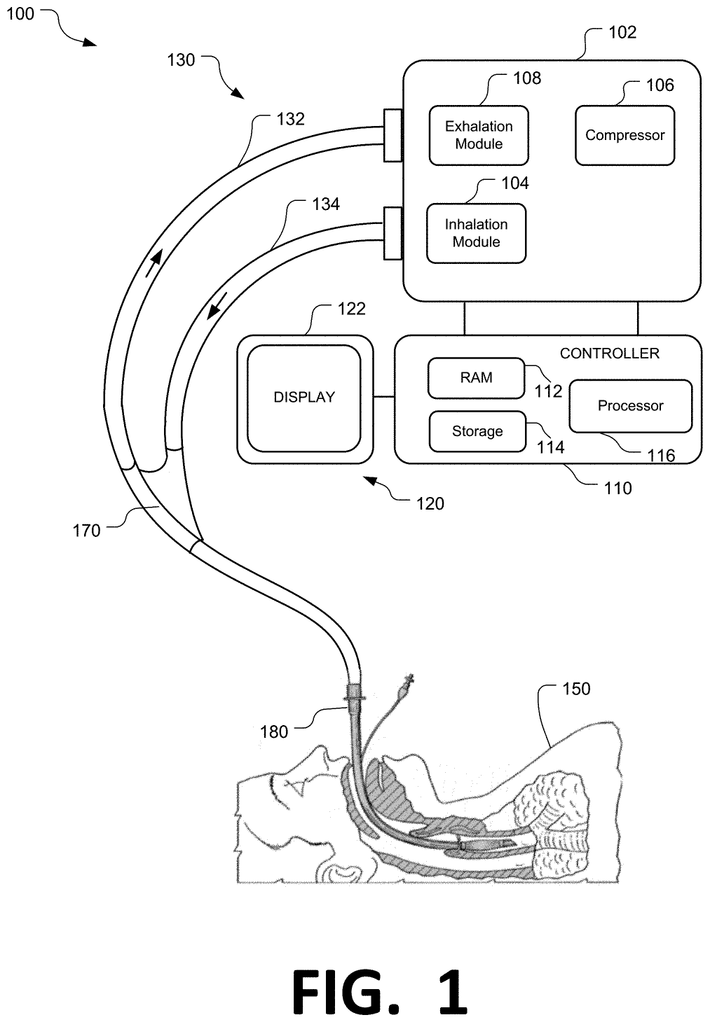

FIG. 1 is a diagram illustrating an example of a medical ventilator 100 connected to a human patient 150. The ventilator 100 may provide positive pressure ventilation to the patient 150. Ventilator 100 includes a pneumatic system 102 (also referred to as a pressure generating system 102) for circulating breathing gases to and from patient 150 via the ventilation tubing system 130, which couples the patient to the pneumatic system via an invasive (e.g., endotracheal tube, as shown) or a non-invasive (e.g., nasal mask) patient interface.

Ventilation tubing system 130 may be a two-limb (shown) or a one-limb circuit for carrying gases to and from the patient 150. In a two-limb example, a fitting, typically referred to as a “wye-fitting” 170, may be provided to couple a patient interface 180 to an inhalation limb 134 and an exhalation limb 132 of the ventilation tubing system 130.

Pneumatic system 102 may have a variety of configurations. In the present example, system 102 includes an exhalation module 108 coupled with the exhalation limb 132 and an inhalation module 104 coupled with the inhalation limb 134. Compressor 106 or other source(s) of pressurized gases (e.g., air, oxygen, and/or helium) is coupled with inhalation module 104 to provide a gas source for ventilatory support via inhalation limb 134. The pneumatic system 102 may include a variety of other components, including mixing modules, valves, sensors, tubing, accumulators, filters, etc., which may be internal or external sensors to the ventilator (and may be communicatively coupled, or capable communicating, with the ventilator).

Controller 110 is operatively coupled with pneumatic system 102, signal measurement and acquisition systems, and an operator interface 120 that may enable an operator to interact with the ventilator 100 (e.g., change ventilation settings, select operational modes, view monitored parameters, etc.). Controller 110 may include memory 112, one or more processors 116, storage 114, and/or other components of the type found in command and control computing devices. In the depicted example, operator interface 120 includes a display 122 that may be touch-sensitive and/or voice-activated, enabling the display 122 to serve both as an input and output device.

The memory 112 includes non-transitory, computer-readable storage media that stores software that is executed by the processor 116 and which controls the operation of the ventilator 100. In an example, the memory 112 includes one or more solid-state storage devices such as flash memory chips. In an alternative example, the memory 112 may be mass storage connected to the processor 116 through a mass storage controller (not shown) and a communications bus (not shown). Although the description of computer-readable media contained herein refers to a solid-state storage, it should be appreciated by those skilled in the art that computer-readable storage media can be any available media that can be accessed by the processor 116. That is, computer-readable storage media includes non-transitory, volatile and non-volatile, removable and non-removable media implemented in any method or technology for storage of information such as computer-readable instructions, data structures, program modules or other data. For example, computer-readable storage media includes RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

Communication between components of the ventilator system or between the ventilator system and other therapeutic equipment and/or remote monitoring systems may be conducted over a distributed network, as described further herein, via wired or wireless means. Further, the present methods may be configured as a presentation layer built over the TCP/IP protocol. TCP/IP stands for “Transmission Control Protocol/Internet Protocol” and provides a basic communication language for many local networks (such as intra- or extranets) and is the primary communication language for the Internet. Specifically, TCP/IP is a bi-layer protocol that allows for the transmission of data over a network. The higher layer, or TCP layer, divides a message into smaller packets, which are reassembled by a receiving TCP layer into the original message. The lower layer, or IP layer, handles addressing and routing of packets so that they are properly received at a destination.

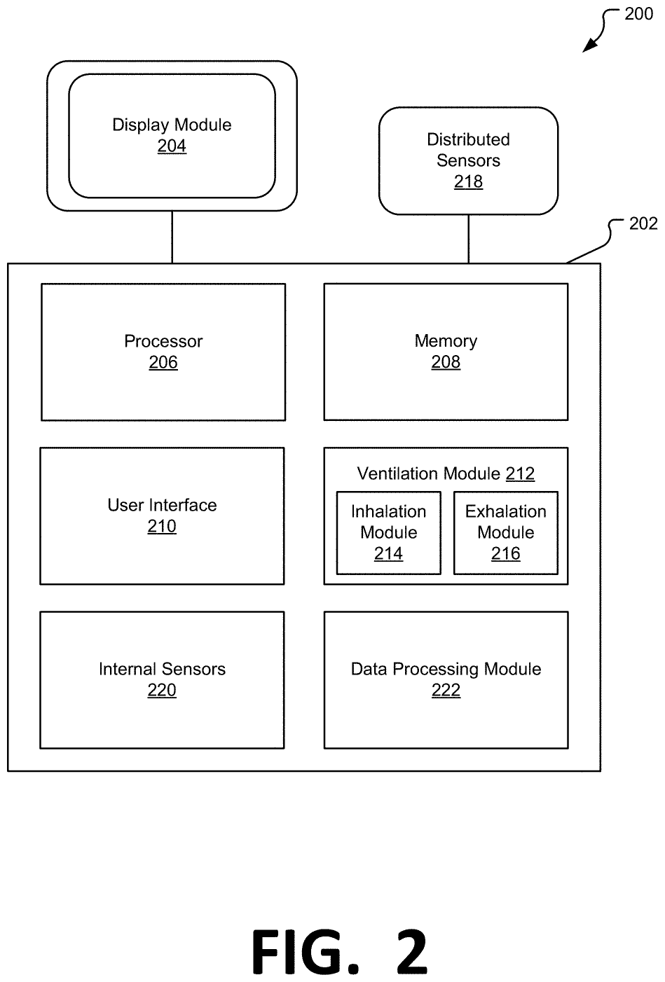

FIG. 2 is a block-diagram illustrating an example of a ventilator system 200. Ventilator system 200 includes ventilator 202 with various modules and components. That is, ventilator 202 may further include, among other things, memory 208, one or more processors 206, user interface 210, and ventilation module 212 (which may further include an inhalation module 214 and an exhalation module 216). Memory 208 is defined as described above for ventilation module 212. Similarly, the one or more processors 206 are defined as described above for one or more processors 206. Processors 206 may further be configured with a clock whereby elapsed time may be monitored by the ventilator system 200.

The ventilator system 200 may also include a display module 204 communicatively coupled to ventilator 202. Display module 204 provides various input screens, for receiving input, and various display screens, for presenting useful information. Inputs may be received from a clinician. The display module 204 is configured to communicate with user interface 210 and may include a graphical user interface (GUI). The GUI may be an interactive display, e.g., a touch-sensitive screen or otherwise, and may provide various windows (i.e., visual areas) comprising elements for receiving user input and interface command operations and for displaying ventilatory information (e.g., ventilatory data, alerts, patient information, parameter settings, modes, etc.). The elements may include controls, graphics, charts, tool bars, input fields, icons, etc. Alternatively, other suitable means of communication with the ventilator 202 may be provided, for instance by a wheel, keyboard, mouse, or other suitable interactive device. Thus, user interface 210 may accept commands and input through display module 204, such as an input parameter for the one-touch ventilation mode. Display module 204 may also provide useful information in the form of various ventilatory data regarding the physical condition of a patient and/or a prescribed respiratory treatment. The useful information may be derived by the ventilator 202, based on data collected by a data processing module 222, and the useful information may be displayed in the form of graphs, wave representations (e.g., a waveform), pie graphs, numbers, or other suitable forms of graphic display. For example, the data processing module 222 may be operative to determine a ventilation settings (otherwise referred to as ventilatory settings, or ventilator settings, or ventilation settings) associated with a one-touch ventilation mode, display information regarding the one-touch ventilation mode, or may otherwise use the one-touch ventilation mode in connection with the ventilator, as detailed herein.

Ventilation module 212 may oversee ventilation of a patient according to ventilation settings. Ventilation settings may include any appropriate input for configuring the ventilator to deliver breathable gases to a particular patient, including measurements and settings associated with exhalation flow of the breathing circuit. Ventilation settings may be entered, e.g., by a clinician based on a prescribed treatment protocol for the particular patient, or automatically generated by the ventilator, e.g., based on attributes (i.e., age, diagnosis, ideal body weight, predicted body weight, gender, ethnicity, etc.) of the particular patient according to any appropriate standard protocol or otherwise, such as may be determined in association with a one-touch ventilation mode. In some cases, certain ventilation settings may be adjusted based on the exhalation flow, e.g., to adjust or improve the prescribed treatment. Ventilation settings may include inhalation flow, frequency of delivered breaths (e.g., respiratory rate, (f), tidal volume (V_{T}), PEEP level, etc.).

Ventilation module 212 may further include an inhalation module 214 configured to deliver gases to the patient and an exhalation module 216 configured to receive exhalation gases from the patient, according to ventilation settings that may be based on the exhalation flow. As described herein, inhalation module 214 may correspond to the inhalation module 104, or may be otherwise coupled to source(s) of pressurized gases (e.g., air, oxygen, and/or helium), and may deliver gases to the patient. As further described herein, exhalation module 216 may correspond to the exhalation module 108, or may be otherwise coupled to gases existing the breathing circuit.

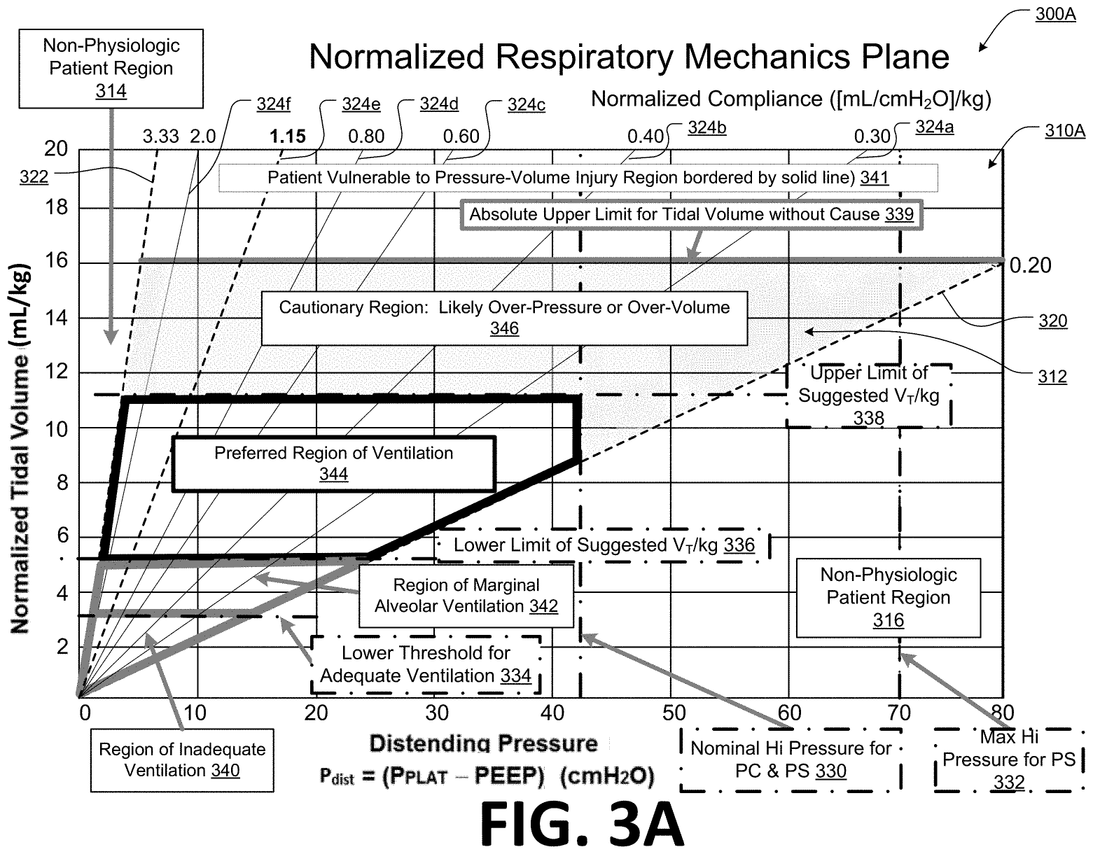

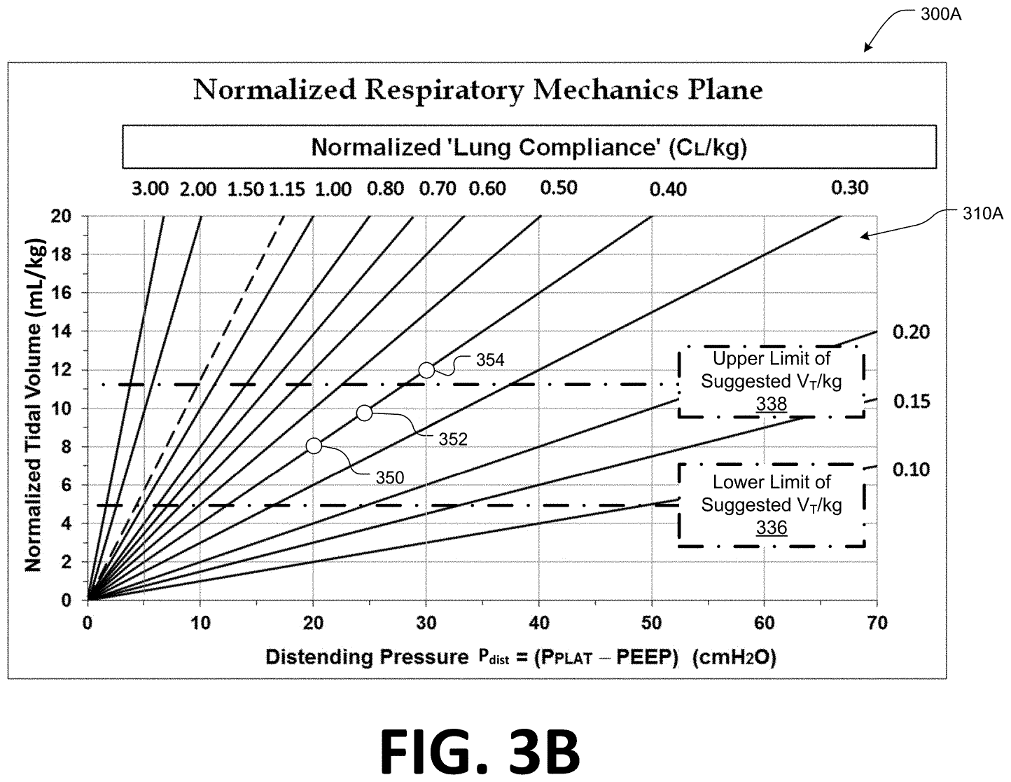

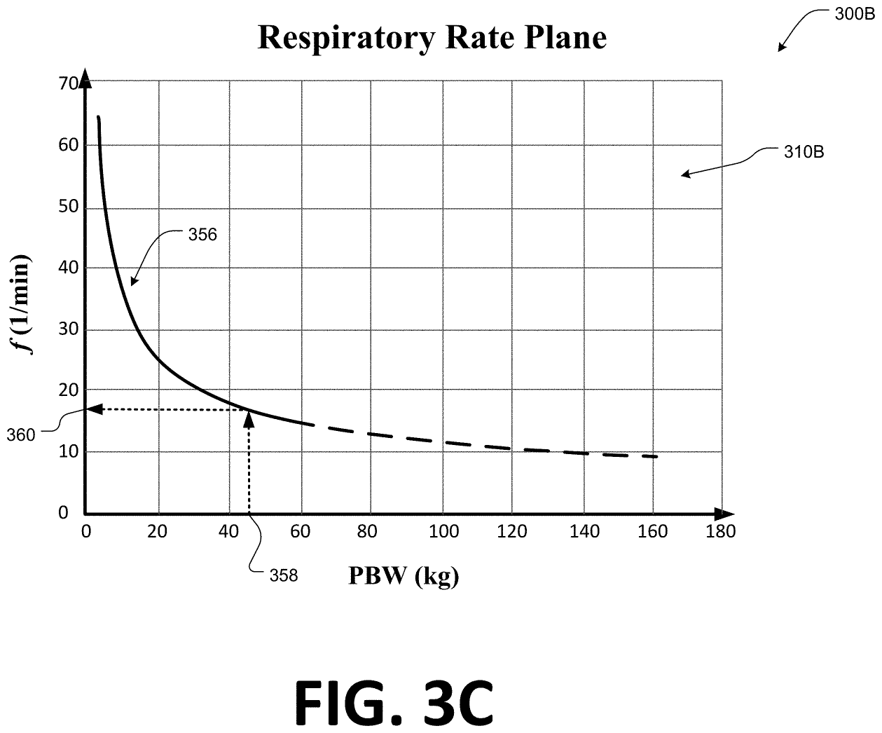

FIGS. 3A-C show charts 300A, 300B illustrating respiratory planes (e.g., normalized respiratory mechanics plane 310A and respiratory rate plane 310B) that may be used with one-touch ventilation mode. For example, one-touch ventilation mode may select and generate initial ventilation settings based on an input parameter by referencing the respiratory planes. For example, the input parameter may be mapped to initial ventilation settings on one or more respiratory plane. As another example, the intrinsic information may be applied to one or more respiratory mechanics plane to generate the initial ventilation settings. One-touch ventilation mode may update the ventilation settings based on patient-specific ventilation data obtained during ventilation. The updated ventilation settings may aim to adjust a patient status point on the plane(s) to maintain or move the patient into a preferred region on the plane, or relative to a preferred point on the plane. Thus, one-touch ventilation mode may use one input parameter (e.g., with one touch) and reference the respiratory planes to select initial and updated ventilation settings. These referenced respiratory planes are further described below as the normalized respiratory mechanics plane and the respiratory rate plane.

FIG. 3A is a chart 300A illustrating an example of a normalized-respiratory-mechanics (NRM) plane 310A. The example NRM plane 310A shown in FIG. 3A provides a visualization of ventilatory mechanics of human patients, normalized by their predicted body weight, as described further below. The NRM plane 310A is defined by distending pressure (P_{dist} or ΔP) on the x-axis, and normalized tidal volume (mL/kg) on the y-axis. Distending pressure is the total pressure applied to the lungs during an inhalation, above the positive end-expiratory pressure (PEEP) level (otherwise referred to herein as a PEEP value, or PEEP). Distending pressure may also be defined as the difference in pressure between the PEEP level and end-inspiratory pressure. In some examples, distending pressure may also be referred to as “drive” pressure. During mechanical ventilation, the distending pressure is the sum of the pressure applied by the ventilator (P_{aw}, or airway pressure) and the pressure applied by the patient's own diaphragmatic efforts (Pₘᵤₛ, or muscle pressure or patient's efforts). That is, P_{dist} equals P_{aw} plus Pₘᵤₛ. If a patient is spontaneously breathing, then the Pₘᵤₛ value will be nonzero. If the patient is not spontaneously breathing (for example, the patient is sedated), then Pₘᵤₛ will be zero, and P_{dist} equals P_{aw}.

Normalized tidal volume is the volume of the breath (in mL), per kilogram (kg) of predicted body weight. Predicted body weight may be an adjusted weight based on a patient's gender and height, rather than an actual weight of the patient. Predicted body weight (PBW, or sometimes referred to as ideal weight) has been found to be a good predictor of the patient's lung size. PBW can be calculated from a patient's gender and height, as height correlates proportionately with PBW. Though PBW is used in this example, the NRM plane 310A may be created based on other indicators of lung size or ideal weight. On the y-axis of the NRM plane 310A, dividing the tidal volume of a breath by PBW normalizes the tidal volume across all patient sizes, enabling patients of very different weights and lung sizes to be placed on the same NRM plane 310A.

The relationship between distending pressure P_{dist} (on the x-axis) and resulting (normalized) tidal volume (V_{T}/kg) of the breath (on the y-axis) can be modeled as a linear relationship, as follows:

P_{dist}=(V_{T}/kg)/(C_{L}/kg) Eq. 1

where (C_{L}/kg) is the normalized lung compliance of the patient's respiratory system. In this model, for a given normalized compliance value C_{L}/kg, increasing the distending pressure (increasing along the x-axis) will produce a normalized tidal volume that increases linearly along an upward line, the line having a slope of (C_{L}/kg). Several such normalized compliance lines are drawn in FIG. 3A, the slope representing exemplary compliance values shown on the NRM plane 310A. These normalized compliance lines radiate out from the origin as compliance lines 324a-f, where the slope of the line is the normalized compliance (C_{L}/kg). Normalized compliance line 324a is associated with a normalized compliance C_{L}/kg of 0.30 (in (mL/cmH₂O)/kg), line 324b is 0.40, line 324c is 0.60, line 324d is 0.80, line 324e is 1.15, and line 324f is 2.0. The boundary lines 320, 322 represent compliance values of 0.20 and 3.33, respectively, which define the physiologic region 312. The physiologic region 312 is defined in this way because normalized compliance values below 0.20 and above 3.33 have not been documented in humans. However, the physiologic region is not limited to these specific boundary lines 320, 322, and can be created with different boundary lines defining different regions.

Compliance is a measure of the lung's ability to stretch or expand. A low compliance value indicates that the lungs are stiff and difficult to stretch. A high compliance value indicates that the lungs expand easily but may not have enough resistance to recoil during exhalation. A healthy compliance value (normalized by kg) may be considered to be about 1.15 (in (mL/cmH₂O)/kg), as indicated by the line 324e. The compliance value for a patient may be obtained (i.e., measured, determined, identified, received, collected, or otherwise acquired) during ventilation as ventilation data. In an example where the normalized tidal volume is known (e.g., as may be selected and/or generated as an initial ventilation setting during one-touch ventilation mode), normalized compliance determined or measured in ventilation data may be used to determine an associated distending pressure. As another example, a known distending pressure and compliance may be used to determine a normalized tidal volume. As a further example, a known normalized tidal volume and known distending pressure may be used to determine normalized compliance. As yet another example, the relationship between normalized tidal volume and distending pressure is defined as a line (e.g., lines 324a-f) with a slope of the normalized compliance (C_{L}/kg) and a zero intercept. Thus, if two out of three of V_{T}, C_{L}, and P_{dist} are known (or the respective normalized values (V_{T}/kg), (C_{L}/kg)), then the third value may be determined.

Accordingly, any matched pair of coordinates for mL/kg and P_{dist} on FIG. 3 locates a unique point on the NRM Plane and that point lies on a line whose slope is C_{L}/kg. Furthermore, all such matched coordinates whose ratio is approximately equivalent (z) will also lie on the normalized compliance line with a slope of normalized compliance (C_{L}/kg). Recognizing that valid estimates for P_{dist} and V_{T} are available, the intersection of orthogonal projections of these two values identifies a probable estimate of the patient's current normalized compliance (C_{L}/kg). A current estimate of a patient's actual compliance (CO is found by multiplying the normalized value (C_{L}/kg) by the patient's estimated PBW.

The scales of the axes on the NRM plane 310A are chosen to span a range of breaths that are physiologically possible in human patients. For example, in FIG. 3A, the x-axis ranges from zero to 300 cmH₂O, and the y-axis ranges from zero to 26 mL/kg. In other embodiments, these ranges can be changed to focus on different areas of breathing or ventilation. The scales of the axes on the NRM plane 310A, the boundary lines 320, 322, lines 324a-f, and the physiologic region 312 were compiled through a thorough review of academic literature to compile pressure, tidal volume, and compliance data from academic studies, research papers, and other publications.

The origin (the intersection of the axes) of the NRM plane 310A represents both the patient and ventilator at rest, except for the ventilator's delivery of a PEEP level. That is, the origin of the x-axis should be set at the value (or level) of PEEP (which could be zero or nonzero). At the origin, Pₘᵤₛ and P_{aw} are both zero, and thus tidal volume (V_{T}) is also zero. The x-axis then shows the distending pressure above the PEEP level.

PEEP is the positive pressure remaining in the lungs at the end of exhalation (positive end-exhalation pressure). In mechanically ventilated patients, PEEP is typically greater than zero, so that some pressure is maintained to keep the lungs inflated and open. The distending pressure along the x-axis is intended to show the amount of pressure that was needed to deliver the resulting tidal volume (on the y-axis). This is an incremental or additional pressure above PEEP, and thus, the x-axis can be set to begin at PEEP instead of at zero. Alternatively, the x-axis can be set to begin at zero, and PEEP can be subtracted from distending pressure, giving an x-axis value of P_{dist} minus PEEP. For example, distending pressure (P_{dist}) may be equal to plateau pressure (P_{PLAT}) minus PEEP. As used herein, the plateau pressure refers to the average pressure applied to the patient's airway and the patient's alveoli at the end of the inspiration phase. The plateau pressure (P_{PLAT}) may be measured at the end of inspiration with an inspiratory hold maneuver by the mechanical ventilator. In another form, distending pressure (P_{dist}) may be equal to airway pressure (P_{aw}) plus patient effort (Pₘᵤₛ).

The NRM plane 310A of FIG. 3A can be interpreted as outlining a pressure-volume space of respiratory activity in humans. In particular, FIG. 3A includes a physiologic region 312, and non-physiologic regions 314 and 316. The physiologic region 312 is a triangular region with boundary lines 320 and 322. As an example, for a distending pressure of 30 cmH₂O (if PEEP is zero, or 30 cmH₂O above PEEP), the physiologic region 312 begins at a normalized tidal volume of about 6 mL/kg. At a distending pressure of 30 cmH₂O (if PEEP is zero), a normalized tidal volume below 6 mL/kg is the non-physiologic region 316. This means that in human patients, a pressure of 30 cmH₂O should deliver a tidal volume greater than 6 mL/kg. As another example, for a tidal volume of 5 mL/kg, the distending pressure in the physiologic region 312 ranges from about 2 to 25 cmH₂O. This means that in human patients, a tidal volume of 5 mL/kg may be produced by distending pressures within a range of about 2 to 25 cmH₂O. On the other sides of the boundary lines 320 and 322 are the non-physiologic regions 314 and 316. These are termed “non-physiologic” because the combinations of distending pressure and tidal volume are not typically found in human patients.

Horizontal and vertical limits may be imposed on the NRM plane 310A to indicate regions of ventilation. For example, in FIG. 3A, the NRM plane 310A is characterized by several different regions and boundaries. Some regions of ventilation on the NRM plane include inadequate ventilation region 340, region of marginal ventilation 342, preferred region of ventilation 344, cautionary region 346 (e.g., the patient is likely to experience over-pressurization or over-volume), and patient vulnerable to injury region 341. These regions of the NRM plane identify when ventilation settings may be injurious to the lung, as well as if the ventilation settings are adequate. The NRM plane 310A includes vertical lines 330 and 332 that indicate nominal and high pressure limits, respectively, for pressure control or pressure support ventilation. Horizontal lines 334, 336, 338, and 339 indicate tidal volume limits. Lower threshold line 334 indicates a threshold below which ventilation is likely inadequate. The inadequate ventilation region 340 of the physiologic region 312 is defined between boundary lines 320 and 322 and lower threshold line 334. In the inadequate ventilation region 340, normalized tidal volume is so low that it is likely to be insufficient to meet the patient's needs for oxygenation and gas exchange. Horizontal line 336 indicates a lower limit of suggested normalized tidal volume for mechanical ventilation of adult patients. The region of marginal ventilation 342 is defined between lines 334 and 336 in the physiologic region 312 (e.g., between boundary lines 320 and 322). The region of marginal ventilation 342 for adults may also be a potentially acceptable region of ventilation for neonatal patients. In the region of marginal ventilation 342, normalized tidal volumes are still potentially too low, but may be acceptable in marginal cases.

The horizontal upper limit of suggested normalized tidal volume line 338 indicates an upper limit of suggested normalized tidal volume for mechanical ventilation. The preferred region of ventilation 344 is bounded by upper limit V_{T} line 338, lower limit V_{T} line 336, nominal high pressure line 330, and the physiologic region boundary lines 320 and 322. Most patients will receive adequate ventilation in the preferred region of ventilation 344.

Horizontal absolute upper limit for tidal volume without cause line 339 indicates an upper limit for normalized tidal volume. The cautionary region 346 is defined below the absolute upper limit for V_{T} line 339 and above the preferred region of ventilation 344 inside the physiologic region 312. In the cautionary region 346, most patients may experience over-pressure or over-volume. The patient vulnerable to injury region 341 is defined above line 339 in the physiologic region. The normalized tidal volumes and distending pressures observed in the patient vulnerable to injury region 341 should not be delivered to human patients, to avoid lung injury.

In an embodiment, the regions of ventilation defined by boundaries in the NRM plane 310A, or that are used for ventilation settings, alarms, or alerts, can be adjusted by a user. For example, any of the boundary lines (such as lines 330, 332, 334, 336, 338, and 339 in FIG. 3A, or any compliance spoke boundaries) can be moved, adjusted, or removed by a user based on a patient's current condition, procedure, or treatment. The ventilator then adjusts its ventilation settings, alerts, or alarms accordingly. For example, the alerts or alarms may be triggered at the positions on the NRM plane 310A desired by the user. An alert or alarm may be any combination of audible, visual, graphic, textual, kinetic, or other messages that inform a clinician to attend to the ventilator and the patient.

In another example, the NRM plane 310A may be used to determine initial ventilation settings of the ventilator. For example, the NRM plane 310A may be used to determine an initial normalized tidal volume or distending pressure. For instance, an input parameter (e.g., PBW) may be used to convert a starting desired normalized tidal volume into a patient-specific tidal volume. As another example, an initial distending pressure may be selected from the NRM plane 310A.

In an example, a ventilator is programmed to adjust a setting in response to such an alert or alarm. For example, the ventilator can adjust a setting by one increment (moving a distending pressure or tidal volume target down by an incremental amount, for example), while continuing to operate the alert or alarm. In an embodiment, a ventilator reduces a calculated pressure target by a set amount in response to an alarm triggered by the ventilator system 200 or NRM plane 310A.

In another embodiment, the NRM plane 310A is used in connection with a closed-loop ventilator system in which the ventilator adjusts settings automatically based on the patient's ventilatory status. The ventilator may also display the patient's current, recently averaged, and/or trending respiratory status on a dashboard display 300 such as illustrated on the NRM plane 310A. A ventilator that is operated by a closed-loop control system (e.g., by receiving ventilation data and updating a patient's position on the NRM plane 310A based on the ventilation data) may continually update a patient's position or point on the NRM plane, as described in FIG. 3B below. The ventilator may display the patient on the NRM plane 310A, enabling the clinician to visualize the patient's ventilatory status and confirm the proper operation of the closed-loop controller to maintain the patient in a safe zone. The processor that executes the program instructions for identifying the patient status and displaying it on the NRM plane 310A may be integrated as part of a closed-loop controller, or may be housed in a different system, such as part of the ventilator, the ventilator display, or a separate processor and display. In another aspect, a feature of the recurring points could be utilized with FIG. 3A, to indicate the trajectory the patient's change as illustrated in FIG. 3B.

FIG. 3B is a chart 300A illustrating a normalized respiratory mechanics plane 310A with provided patient temporal status points 350, 352, 354. In an example, an individual patient's normalized tidal volume and distending pressure is plotted on the NRM plane 310A to provide a characterization of the patient's respiratory status in relation to a region of the NRM plane 310A. For example, a graphical marker such as circle is placed at a point 350, 352, 354 (or location) on the NRM plane 310A corresponding to the patient's most recent breath (or average of recent breaths). Alternatively, the patient's point on NRM plane 310A may not be displayed by the ventilator, but may still be used by the ventilator as an evaluation of the region of ventilation of the patient. Specifically, FIG. 3B illustrates a representation (shown as a point 350, 352, 354) of single breaths (or averages of recent breaths) whose normalized tidal volume and distending pressure fall along a normalized lung compliance (C_{L}/kg) of 0.40 (mL/cmH₂O)/kg, with tidal volumes ranging between 8 mL/kg and 12 mL/kg and distending pressure ranging from 20 cmH₂O to 30 cmH₂O. In this example, the patient's lung compliance remains constant, while normalized tidal volume and distending pressure change with points 350, 352, 354. Points 350 and 352, as shown, fall in the preferred region of ventilation 344, between upper limit line 338 and lower limit line 336. Point 354 falls in the cautionary region 346. When the ventilator is operating in one-touch ventilation mode, the ventilation settings may be changed to move point 354 back into the preferred region of ventilation 344, similar to points 350 and 352. Although FIG. 3B shows a patient with a constant compliance, it should be appreciate that the compliance value may change for a patient from time to time.

The connection between sequential points indicates rate of change and a notification may be provided by the ventilator to the clinician based on this rate of change. At the end of each interval, the ventilator may analyze the patient's sensor data and indicate the patient's location on the NRM plane 310A. Points 350, 352, 354 may each be plotted on the NRM plane 310A. In some examples, each point 350, 352, 354 is time stamped on the chart. The points 350, 352, 354, illustrated in FIG. 3B, indicate that the compliance remained constant but the patient's normalized tidal volume and distending pressure increased Given that the sequential values for normalized tidal volume, normalized distending pressure, and compliance could change in any of several logical trajectories, a temporal indicator on the NRM plane 310A can apprise a clinician of the patient's status.

FIG. 3C is a chart 300B illustrating a respiratory rate (RR) plane 310B that shows the relationship 356 between PBW and respiratory rate (f). The RR plane 310B may be used to determine an initial respiratory rate for initial settings of the ventilator. For instance, an input parameter 358 may be associated with a respiratory rate 360 based on the relationship 356. As an example, based on the RR plane 310B, an initial respiratory rate setting for ventilation may be 18 breaths per minute for a patient having a PBW of 45 kg.

Although the RR plane 310B shows the input parameter 358 as PBW, other inputs may be used to determine the respiratory rate. For example, the relationship 356 may be based on, or influenced by, age, ethnicity, etc., which may each individually estimate a respiratory rate, or may be used in combination. Although the RR plane 310B may be used to determine initial ventilation settings associated with respiratory rate, the respiratory rate and the RR plane 310B may be adjustable or adaptive based on other measured or determined parameters. For example, the respiratory rate may be based on tidal volume, a lung condition, breath type strategy, external monitors such as a blood pressure monitor, oximeter, etc. For example, if the patient is breathing spontaneously, the respiratory rate may be adjusted or adapted to match the spontaneous breathing rate of the patient. As another example, the respiratory rate may have minimum or maximum thresholds. In an example, the respiratory rate may not drop below a minimum threshold and/or may not exceed a maximum threshold, despite adjustments associated with obtained ventilation data.

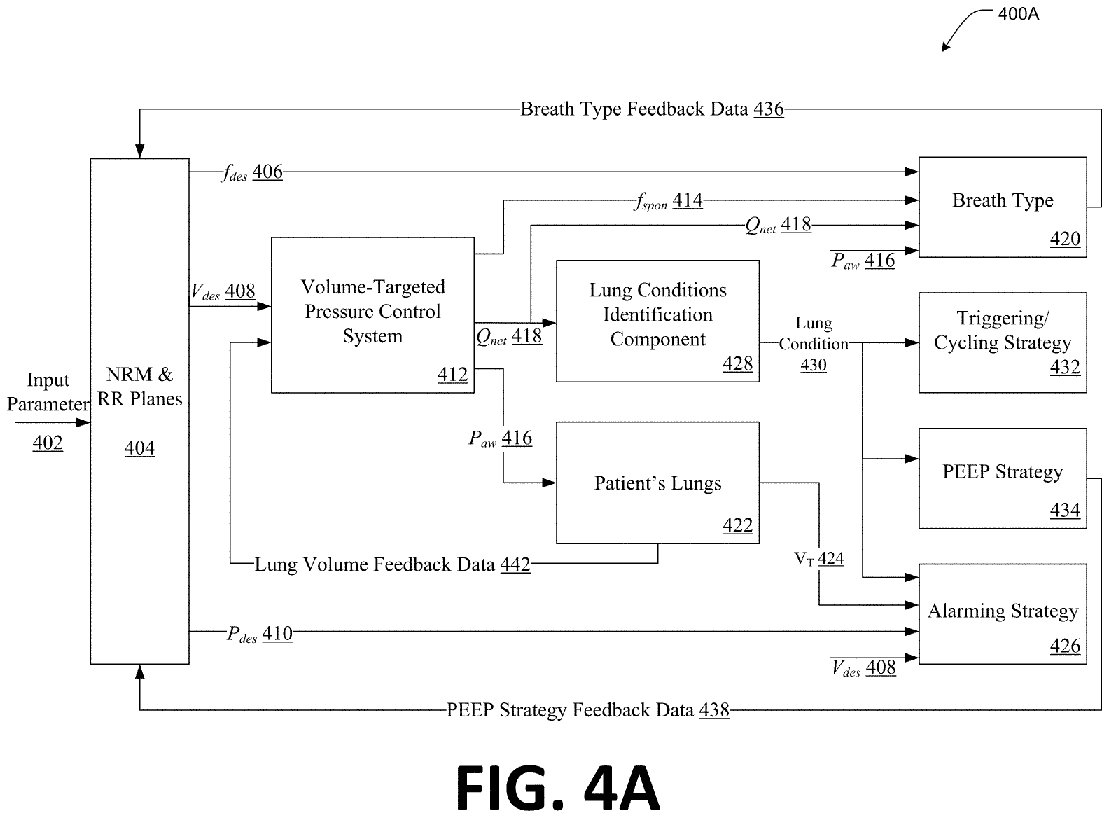

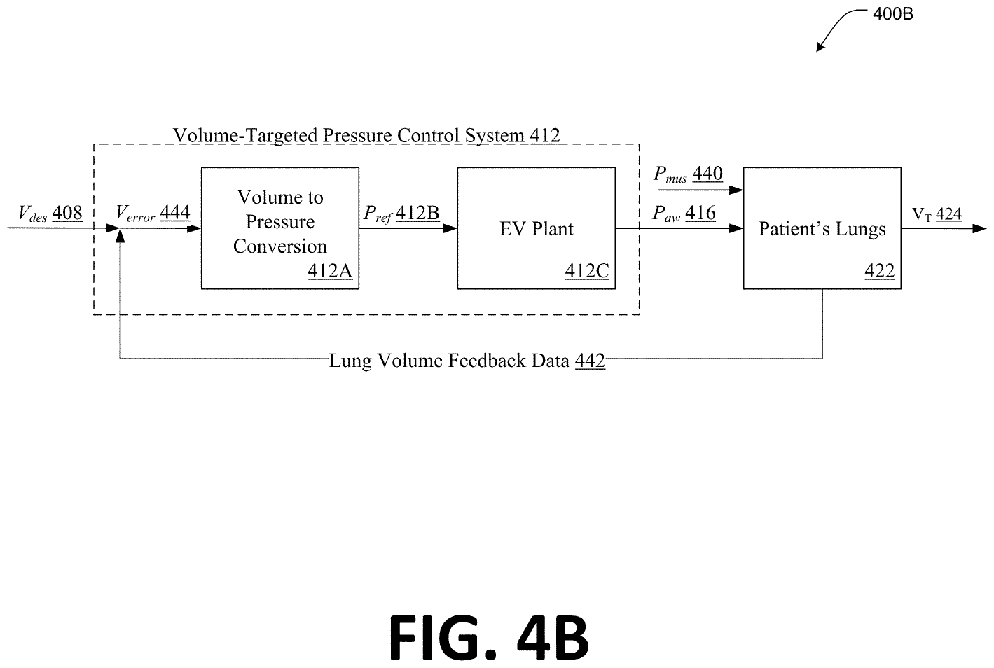

FIG. 4A is block diagram illustrating a schematic flowchart 400 for one-touch ventilation mode. One-touch ventilation mode may begin when the ventilator receives an input parameter 402. This one-touch ventilation mode may be automatically initiated upon receiving an input parameter 402, or may additionally receive an indication of mode selection and/or mode initiation. The input parameter 402 may be received from a clinician or user, may be measured or derived from external sensors, or may be measured or determined from the ventilator prior to or during ventilation. For example, the input parameter 402 may be intrinsic patient information, such as PBW, age, ethnicity, or other intrinsic information of a patient. As a further example, the input parameter 402 may be information obtained from an external sensor, such as oxygen saturation from a pulse oximeter, partial pressure of end-tidal CO₂, temperature from a thermometer, blood pressure from a blood pressure monitor, scale, etc. The external sensor may be communicatively coupled with the ventilator. The input parameter 402 may include a plurality of parameters, such as intrinsic information (e.g., PBW, age, ethnicity, etc.) and measured patient data (e.g., oxygen saturation, partial pressure of end-tidal CO₂, blood pressure, etc.).

Based on the input parameter 402, the ventilator may reference NRM and RR planes 404 (such as NRM plane 310A and RR plane 310B) to determine a desired respiratory rate (f_{des}) 406, a desired tidal volume (V_{des}) 408, and/or a desired distending pressure (P_{des}) 410. The desired respiratory rate 406, desired tidal volume 408, and desired distending pressure 410 may be automatically selected from a preferred region of ventilation (such as preferred region of ventilation 344). Alternatively, a user or clinician may select the desired respiratory rate 406 from the RR plane and/or the desired tidal volume 408 and desired distending pressure 410 from the NRM plane. In an example where the ventilator automatically selects desired respiratory rate 406 from the RR plane, the ventilator may use a relationship identified or determined between the input parameter 402 and the respiratory rate, such as relationship 356.

The relationship 356 may be based on a function. For example, as shown, relationship 356 between the input parameter 402 (e.g., PBW) and respiratory rate 406 on the RR plane may be a negative exponential function. As shown, a lower PBW is associated with a higher respiratory rate 406, and a high PBW is associated with a lower respiratory rate 406. There may be a minimum respiratory rate associated with the relationship 356. As an example, the minimum respiratory rate on the RR plane may be between 1-8 breaths per minute, as may be associated with an asymptote in the function. Alternatively, the function may be piecemeal and may have a relative and/or absolute minimum associated with a minimum respiratory rate. There may not be a maximum respiratory rate associated with the functional relationship 356. For example, the functional relationship 356 may have an asymptote approaching infinity at a PBW of zero. Alternatively, the functional relationship 356 may have a maximum respiratory rate associated with any PBW below a specified value. It should be appreciated that, although a negative exponential function is shown by the relationship 356, any function may be used to determine a respiratory rate 406 from one or more input parameters 402.

In an example where the ventilator automatically selects desired tidal volume 408 and desired distending pressure 410 from the NRM plane, the automatic selection may also be based on additional constraints. The additional constraints may be associated with the normalized tidal volume. For example, the ventilator may automatically select a desired tidal volume 408 based on the lowest normalized tidal volume of the preferred region of ventilation, such as the lower limit line 336 of normalized tidal volume, to prevent damaging the lungs of the patient. The ventilator may then select a desired distending pressure 410 based on the determined compliance for the patient and the selected desired tidal volume 408. As another example, the desired tidal volume 408 may be selected in the center of the range of normalized tidal volumes in the preferred region of ventilation.

The desired respiratory rate 406, desired tidal volume 408, and desired distending pressure 410 may be updated from time to time based on measured, determined, or received parameters, data, or information. After the desired respiratory rate 406, desired tidal volume 408, and desired distending pressure 410 are selected or determined, the ventilator may automatically generate and set initial ventilation settings based on these desired values. Additionally or alternatively, the ventilator may display these desired values and/or graphically display a desired point or desired region or desired line (such as when compliance is known) on the NRM and/or the RR planes 404, representing the desired values. As another example, the ventilator may wait for verification by a clinician prior to setting initial ventilation settings.

Additionally, the desired respiratory rate 406, desired tidal volume 408, and desired distending pressure 410 may be used or associated with a variety of ventilation algorithms and strategies. For example, one-touch ventilation mode may implement a volume-targeted pressure control system 412 and/or lung condition identification component 428. As a further example, one-touch ventilation mode may include one or more strategies, such as breath type strategy 420, alarming strategy 426, triggering/cycling strategy 432, PEEP strategy 434, etc. As an example, the desired tidal volume 408 may be used or associated with a volume-targeted pressure control system 412. Aspects of the volume-targeted pressure control system 412 are further described in FIG. 4B. The volume-targeted pressure control system 412 may derive or determine a spontaneous respiratory rate (fₛₚₒᵣₜ) 414, an airway pressure (P_{aw}) 416, and a net flow (Qₙₑₜ) 418.

The ventilator may determine a breath type strategy 420 using at least one of the following patient parameters: desired respiratory rate 406, spontaneous respiratory rate 414, airway pressure 416, and net flow 418. The breath type strategy 420 may select or determine a breathing type of the patient. For example, the breathing type may be a mandatory breath (e.g., as delivered in a mandatory mode) or a spontaneous breath (e.g., as delivered in a spontaneous mode). In an example, the breath type strategy may change based on the patient parameters. For example, the ventilator may switch from mandatory breath to spontaneous breath, or vice versa, if a change in the breathing efforts from the patient is detected. In an example, the ventilator may begin initial ventilation settings in mandatory breath and may switch to spontaneous breath if patient effort is detected. In another example, the ventilator may switch from spontaneous breath to mandatory breath if a missed-triggering event occurs and/or if the spontaneous respiratory rate 414 drops below a minimum threshold or a threshold below the desired respiratory rate 406. For example, a threshold may be based on an error tolerance above and/or below the desired respiratory rate. As another example, maximum and/or minimum thresholds may be associated with respiratory rate on the RR plane for any desired respiratory rate.

The breath type strategy 420 may provide breath type feedback data 436 to the NRM and RR planes 404. In an example, the breath type strategy 420 may apply a spontaneous respiratory rate 414 in a spontaneous mode and send that information in the breath type feedback data 436 to the RR plane. The ventilator may use the breath type feedback data 436 to compare the spontaneous respiratory rate 414 produced by the breath type strategy with the RR plane to determine if the spontaneous respiratory rate 414 exceeds a minimum or maximum respiratory rate threshold of the RR plane, or as otherwise determined by the ventilator. In an example, if the spontaneous respiratory rate exceeds a minimum or maximum respiratory rate threshold, then the breath type strategy may be switched to mandatory mode. In another example, the minimum and maximum thresholds may be compared or referenced at the breath type strategy 420 determination, prior to sending breath type feedback data 436.

In a further example, the RR plane may be referenced based on a change in the input parameter(s) 402. For example, a point representing a patient on the RR plane may change or update, based on changes in other patient parameters (e.g., oxygen saturation, pulse, body temperature, etc.). As a patient data point moves along the RR plane, the desired respiratory rate 406 may also change. A change in the desired respiratory rate 406 from the RR plane may influence the respiratory rate in mandatory mode, as well as influence the respiratory rate thresholds in spontaneous mode. Thus, the ventilator may continually update respiratory rate of the ventilation settings in one-touch ventilation mode based on changes in patient parameters. In an example, the RR plane may not change over time, while a point representing a patient on the plane may change.

Based on the net flow 418, the ventilator may also perform lung condition identification component 428. The lung condition identification component 428 may classify the patient's lung condition 430 in a category, such as obstructive type, restrictive type, or normal. Obstructive type lung condition 430 may include the patient having difficulty exhaling all of the air from the lungs. For example, obstructive type lung condition 430 may include COPD and asthma. Patients with restrictive type lung condition 430 may have difficulty fully expanding the lungs with air, such as ARDS. The lung condition identification component 428 may be associated with an expiratory time constant (τₑₓₚ) which is the product of compliance and resistance during exhalation phase. The expiratory time constant may aid in identifying a lung condition 430 and its severity. For example, a ventilated patient with a normal lung may have an expiratory time constant between 0.5 and 0.7 seconds. As an example, for a patient with ARDS, the expiratory time constant may be between 0.3 and 0.5 seconds. The expiratory time constant may be even shorter than the aforementioned range for a patient with more severe ARDS, which may indicate low compliance and a small volume of an aerated lung. As another example, in patients with chest-wall stiffness such as kyphoscoliosis, the expiratory time constant may be between 0.15 and 0.25 seconds. In yet another example, an expiratory time constant that is longer than a normal patient (e.g., longer than 0.7 seconds) may indicate COPD and asthmatic patients. In a further example, patients with severe bronchospasm may have an expiratory time constant that could be as long as, or exceed, 3.0 seconds.

The expiratory time constant (τₑₓₚ) may be determined based on the following relationship:

Qₙₑₜ=Qₚₑₐₖ*e^{-t}^{_{elapsed}}^{/τ}^{ₑₛₚ} Eq. 2

where t_{elapsed} is the time elapsed from the onset of an exhalation phase of a breath, and Qₚₑₐₖ is the peak net flow during the exhalation phase. From Eq. 3, the expiratory time constant (τₑₓₚ) may be derived as:

$${τ}_{e x p} = \frac{- {t}_{elapsed}}{ln ( {Q}_{net} ( {t}_{elapsed} ) / {Q}_{peak} )} \qquad Eq . 3$$

where Qₙₑₜ(t_{elapsed}) is the net flow at the time elapsed of the exhalation phase. Thus, the expiratory time constant (τₑₓₚ) may be determined based on net flow 418, which may be associated with a lung condition 430 by the lung condition identification component 428. The lung condition identification component 428 may be changed or updated from time to time based on updated desired respiratory rate 406, desired tidal volume 408, and/or desired distending pressure 410. Additionally, based on the identified lung condition 430, protective measures may be applied to prevent ventilator-induced injury to the patient. For example, the lung condition 430 may be associated with a maximum tidal volume and/or a maximum distending pressure (above either of which an injury may occur). In an example, if the desired tidal volume 408 and/or the desired distending pressure 410 is above the maximum, based on the lung condition 430, the desired tidal volume 408 and/or the desired distending pressure 410 may be reduced.

As an example, the lung condition 430 may be determined by the lung condition identification component 428 by measuring or determining time elapsed from the onset of an exhalation phase of a breath (t_{elapsed}), the peak net flow during the exhalation phase (Qₚₑₐₖ), and the net flow at the time elapsed of the exhalation phase (Qₙₑₜ(t_{elapsed})). These measured or determined values may then be used to determine the expiratory time constant (τₑₓₚ) based on the relationship described above in Eqn. 4. The lung condition identification component 428 may compare the expiratory time constant (τₑₓₚ) to one or more time constant thresholds indicative of a different lung condition 430. Based on the comparison, the lung condition 430 for the patient may be identified. For example, an expiratory time constant (τₑₓₚ) of 0.2 seconds may be identified as a kyphoscoliosis lung condition 430. As another example, an expiratory time constant (τₑₓₚ) of 0.3 seconds may be identified as an ARDS or severe ARDS lung condition 430. As a further example, an expiratory time constant (τₑₓₚ) of 0.6 seconds may be identified as a normal lung condition 430. In yet another example, an expiratory time constant (τₑₓₚ) of 1.0 may be identified as a COPD or asthmatic lung condition 430. In a further example, an expiratory time constant (τₑₓₚ) of 2.8 seconds may be identified as a severe bronchospasm lung condition 430.

Based on the identified patient's lung condition 430, the one-touch ventilation mode may select a triggering/cycling strategy 432. In some triggering/cycling strategies 432, a patient's inspiratory trigger is detected based on the magnitude of deviations (deviations generated by a patient's inspiratory effort) of a measured parameter from a determined baseline. In examples, a triggering strategy or trigger type may be a flow trigger type, pressure trigger type, signal distortion trigger type, synchronized trigger type, etc. In further examples, a cycling strategy or cycle type may be a flow cycle type, pressure cycle type, etc. For example, in a flow triggering strategy or flow trigger type, the patient's inspiration effort is detected when the measured patient exhalation flow value drops below a flow baseline (i.e., the base flow) by a set amount (based on the triggering sensitivity). In a pressure triggering strategy or pressure trigger type, the patient's inspiration effort is detected when the measured expiratory pressure value drops below a pressure baseline (for example, the set PEEP level) by a set amount (based on triggering sensitivity). Another parameter that can be used for a triggering strategy trigger type is a derived signal, such as an estimate of the intrapleural pressure of the patient and/or the derivative of the estimate of the patient's intrapleural pressure. The term “intrapleural pressure,” as used herein, refers generally to the pressure exerted by the patient's diaphragm on the cavity in the thorax that contains the lungs, or the pleural cavity. The derivative of the intrapleural pressure value will be referred to herein as a “Psync” value that has units of pressure per time. An example of triggering and cycling based on the Psync value is provided in U.S. patent application Ser. No. 16/411,916 (“the '916 Application”), titled “Systems and Methods for Respiratory Effort Detection Utilizing Signal Distortion” and filed on May 14, 2019, which is incorporated herein by reference in its entirety. That triggering strategy discussed in the '916 Application is referred to herein as the “signal distortion” triggering strategy or “signal distortion” trigger type. As discussed in the '916 Application, the signal distortion triggering strategy may operate on the Psync signal or other signals, such as flow or pressure.

Each type of triggering strategy or trigger type (e.g., flow triggering, pressure triggering, signal distortion triggering, etc.) has different benefits and drawbacks for different types of patients. In addition, various ventilation settings may be adjusted to better suit each type of patient. By selecting the best-suited triggering strategy or trigger type and the best-suited settings within that triggering strategy or trigger type, patient synchrony may be improved, resulting in a decrease in patient discomfort.

Identifying the proper triggering strategy or trigger type may be based on the patient's lung condition 430. For example, the ventilator may automatically select a particular triggering type and/or cycling type that synchronizes the breathing cycle with the patient's natural breathing pattern. As an example, if the patient's lung condition 430 is obstructive the ventilator may select a mode associated with a signal distortion triggering strategy or signal distortion trigger type. In another example, if the patient's lung condition 430 is restrictive, the ventilator may automatically select a triggering type and cycling type based on flow. In yet another example, if the patient's lung condition 430 is normal but otherwise has an airflow limitation that may be caused by high respiratory rate or short exhalation time, the ventilator may select a synchronized triggering strategy or synchronized trigger type with a flow cycling strategy or flow cycle type.

Additionally or alternatively, the patient's lung condition 430 may be associated with a PEEP strategy 434. The ventilator may use a PEEP strategy 434 or a PEEP protocol to help keep the patient's alveoli open and prevent small airway closure. In an example, the ventilator may have a minimum threshold for PEEP (i.e., a minimum threshold for a PEEP level), such as 5.0 cmH₂O. In another example of a PEEP strategy or PEEP protocol, PEEP may be increased, as required or desired, based on the patient's lung condition. For example, a PEEP strategy 434 or PEEP protocol to support oxygenation in patients with severe hypoxemia, may increase PEEP between 15 and 20 cmH₂O. In another example, a PEEP strategy 434 or a PEEP protocol may be associated with diffusing lung disease such as ARDS, pulmonary edema, diffuse alveolar, etc. In an aspect, one-touch ventilation mode may have a default PEEP strategy or default PEEP protocol, such as 5.0 cmH₂O. The ventilator may continually monitor the patient's lung condition 430 to adjust the PEEP strategy 434 or PEEP protocol.

Additionally, a change in the lung condition 430, may change the point (such as points 350, 352, 354) associated with the patient on the referenced NRM and/or RR planes 404 and/or update desired ventilation parameters, to result in a change of ventilation settings. For example, a change in lung condition may change the triggering strategy or cycling strategy, which may then change a patient's point on the reference planes and/or change the desired ventilation parameters (e.g., desired respiratory rate 406, desired tidal volume, 408, and/or desired distending pressure 410). As another example, the PEEP strategy 434 may send PEEP strategy feedback data 438 to the NRM and RR planes 404. The PEEP strategy feedback data 438 may be associated with a change in the normalized distending pressure of the point on the plane(s) associated with the patient. In yet another example, the upper limit and lower limit on a preferred region of ventilation (e.g., upper limit 338 and lower limit 336 of preferred region of ventilation 344) may change based on obtained ventilation data and strategies (e.g., alarming strategy, triggering strategy, cycling strategy, PEEP strategy, etc.). In a further example, a change in desired ventilation parameters (as may be caused by a change in lung conditions) may also update various ventilation data and strategies, such as alarming strategy, triggering/cycling strategy, PEEP strategy, etc. Thus, the one-touch ventilation mode may have continuous updating of desired ventilation parameters, ventilation data, and strategies, as may each be impacted by a change in the other. In an example, the NRM plane and/or RR plane may not change over time, while a point representing a patient on the NRM plane and/or RR plane may change.

An alarming strategy 426 may be based on one or more parameters, such as the airway pressure 416, a lung volume (V_{T}) 424, the desired tidal volume 408, and the desired distending pressure 410. As further described with respect to FIG. 4B, the lung volume may be determined based on the airway pressure 416, and patent's efforts (Pₘᵤₛ) 440. The alarming strategy 426 may have different categories of alarms, such as protective alarms and informative alarms. As an example, protective alarms may be associated with a risk of ventilator-induced injury to the patient.

For example, a protective alarm may be associated with lung conditions of the patient that may be determined or identified at lung condition identification component 428. As an example, if the patient's lungs are identified as restrictive type, tidal volume and distending pressure may be closely monitored to prevent lung injury. For example, an alarm may be associated with a threshold tidal volume and/or a threshold distending pressure. As a further example, if the patient's lung is identified as restrictive type, additionally or alternatively, the desired tidal volume 408 may be incrementally decreased to a safe level, such as 4.0 mL/kg, to ensure the static or plateau pressure (P_{PLAT}) is below 30.0 cmH₂O. In another example, if the patient's lung is identified as a restrictive type, the airway pressure 416 and lung volume 424 may also be continuously monitored to prevent injury to the patient's lungs. An alarm may trigger if the airway pressure 416 and/or lung volume 424 exceed threshold values. The alarming strategy 426 or ventilation settings may be changed or updated from time to time based on updated desired respiratory rate 406, desired tidal volume 408, and/or desired distending pressure 410. Additionally or alternatively, an alarm may be based on a preferred region of ventilation. For example, an alarm may be issued if a patient exceeds a threshold associated with a boundary of a preferred region of ventilation (e.g., preferred region of ventilation 344). In this example, an alarm may be at one or more boundaries, inside of one or more boundaries, or outside of one or more boundaries of the preferred region of ventilation, as may be pre-set or pre-determined.

FIG. 4B is a block diagram illustrating a volume targeted pressure control system 400B, shown as a subset of the schematic flowchart 400A of one-touch ventilation mode shown in FIG. 4A. The volume-targeted pressure control system 412 may be a closed loop system that includes a volume-to-pressure conversion 412A to convert the desired tidal volume 408 to a reference pressure 412B that is then processed by the exhalation valve assembly and control system 412C (otherwise referred to as the EV plant 412C) to estimate airway pressure 416. The airway pressure 416 together with the patient's effort 440 may then be fed to the patient's lungs 422 to allow the ventilator to estimate lung volume 424 (e.g., the volume of air delivered into the patient's lungs). The closed loop system then provides lung volume feedback data 442 that may be used to compare the lung volume 424 with the desired tidal volume 408. A volume error 444 is determined based on the comparison between the desired tidal volume 408 and the lung volume 424 (e.g., by subtracting the lung volume 424 from the desired tidal volume 408). The volume error 444 may be used to adjust ventilation settings to proportionally change the airway pressure 416 to minimize the volume error 444. For example, one-touch ventilation mode may generate ventilation settings to influence the airway pressure 416 by changing the inhalation flow and/or exhalation pressure. As an example, if the volume error 444 indicates that the lung volume 424 is lower than the desired tidal volume, then the one-touch ventilation mode may generate updated ventilation settings to increase the airway pressure 416 and thus increase the lung volume 424 (e.g., increase inhalation flow and/or increase exhalation pressure). As another example, if the volume error 444 indicates that the lung volume 424 is higher than the desired tidal volume, then the one-touch ventilation mode may generate updated ventilation settings to decrease the airway pressure 416 and thus decrease the lung volume 424 (e.g., decrease inhalation flow and/or decrease exhalation pressure).

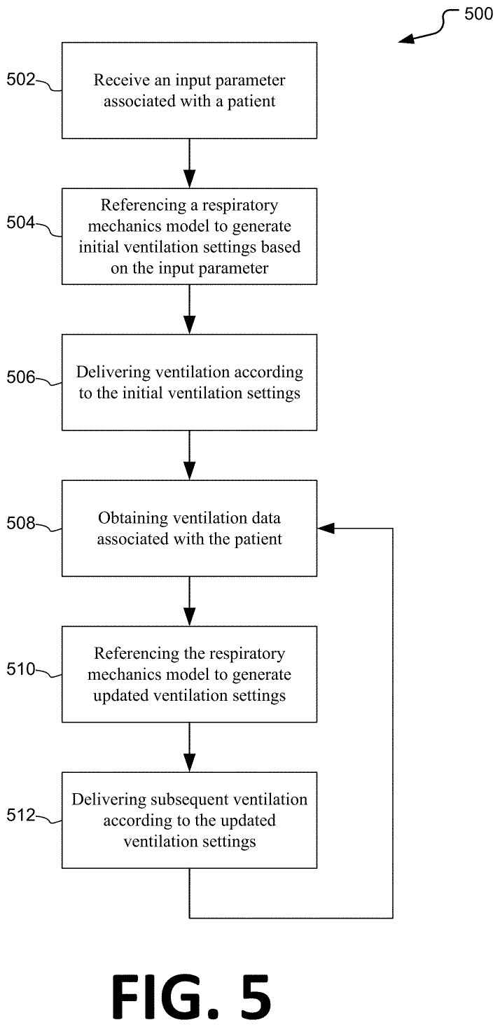

FIG. 5 is a flowchart illustrating a method 500 for one-touch ventilation mode. The method 500 begins at operation 502 where a mechanical ventilator receives an input parameter associated with a patient. The input parameter, as described herein, may be intrinsic information of a patient or information measured or determined from external sensors. There may be a plurality of input parameters. Alternatively, one-touch ventilation mode may be capable of functioning with only one input parameter.

At operation 504, one-touch ventilation mode references a respiratory mechanics model to generate initial ventilation settings based on the input parameter. In an example, a respiratory mechanics model may include the NRM plane and/or RR plane described herein. As further described herein, referencing the respiratory mechanics model may include mapping the input parameter to ventilation settings on the plane, or applying the input parameter to the plane to generate ventilation settings. As an example, the initial ventilation settings may be based on a desired ventilation parameter obtained from the respiratory mechanics plane, such as desired tidal volume, desired respiratory rate, and/or desired distending pressure. As described herein, the respiratory mechanics plane may be an NRM plane and/or an RR plane. The respiratory mechanics plane that is referenced may depend on the type of input parameter received. In an example where the input parameter is intrinsic information, one-touch ventilation mode may reference both the NRM plane and the RR plane to determine at least one desired ventilation parameter. In a specific example where the input parameter is PBW, the ventilator may reference the NRM plane to determine a desired tidal volume (e.g., select a normalized tidal volume from the NRM plane and then determine a patient-specific, desired tidal volume based on the PBW), and a desired respiratory rate from the RR plane (e.g., use an established relationship between PBW and respiratory rate from the RR plane). The ventilator may generate ventilation settings based on the input parameter.

At operation 506, the ventilator may deliver ventilation according to the initial ventilation settings determined at operation 504. For example, the ventilator may adjust or change inhalation flow and/or exhalation pressure. As an example, tidal volume may be changed until the patient tidal volume (or delivered lung volume) approximately equals a desired tidal volume determined from referencing the NRM plane (e.g., mapping the input parameter to initial ventilation settings on the NRM plane, or applying the input parameter to the NRM plane to generate the initial ventilation settings).

At operation 508, the ventilator may obtain, measure, determine, identify, or acquire ventilation data associated with the patient. As described herein, ventilation data may be any information determined by the ventilator while ventilating the patient (such as spontaneous breath rate, expiratory time constant, PEEP, patient effort, airway pressure, inhalation flow, exhalation flow, inhalation pressure, exhalation pressure, interface pressure, etc.). The ventilation data may be associated with a determined ventilation strategy, such as breath type strategy, alarming strategy, triggering strategy, cycling strategy, PEEP strategy, etc.

At operation 510, the ventilator may reference (as further described herein) the respiratory mechanics plane to generate updated ventilation settings based on the ventilation data. For example, as ventilation data is received (e.g., from external sensors or from the ventilator) the respiratory mechanics plane(s) may be referenced to determine additional desired ventilation parameters, such as a desired distending pressure, lung compliance, maximum and minimum tidal volume and distending pressure thresholds, spontaneous respiratory rate, etc. Additionally or alternatively, the respiratory mechanics plane may be referenced to compare a current patient status point (e.g., status points 350-354, based on the ventilation data) relative to a preferred region of ventilation on the respiratory mechanics plane (e.g., preferred region of ventilation 344). The updated ventilation settings may compensate or adjust for an undesired shift in the patient status point over time, aim to place the patient status point in the preferred region of ventilation, and/or adjust the patient status point relative to a threshold, line, or boundary associated with the respiratory mechanics plane.

At operation 512, the ventilator delivers subsequent ventilation based on the updated ventilation settings. Updated ventilation settings may be delivered similarly to operation 506.

As required or desired, the method 500 for one-touch ventilation mode may repeat operations 508 through 512 as additional ventilation data is received by the ventilator, and/or when ventilation data changes. For example, the ventilator may receive additional or updated ventilation data during the course of ventilation that may change or update the desired ventilation parameter(s) (e.g., when referencing the respiratory mechanics plane) and/or require the ventilator to update the ventilation settings to continue to update ventilation to shift a patient status point relative to the respiratory mechanics plane. For example, the ventilator may determine a compliance of the patient, which may determine a patient status point one of tidal volume or distending pressure is unknown. In an example, a change in delivered lung volume, lung condition, or spontaneous respiratory rate may change a patient status point without changing desired ventilation parameters determined from referencing the respiratory mechanics plane. In this example, the ventilator may generate new ventilation settings to minimize an error between delivered and desired ventilation parameters, without changing the desired ventilation parameters. As another example, the ventilator may determine a change in PEEP strategy, which may change the desired distending pressure as referenced from the respiratory mechanics plane. As a further example, the spontaneous respiratory rate may change, which may change the desired respiratory rate from the RR plane. Additionally or alternatively, changes in lung conditions may change one or more desired ventilation parameters. Additionally, one-touch ventilation mode may automatically generate and deliver the ventilation settings.

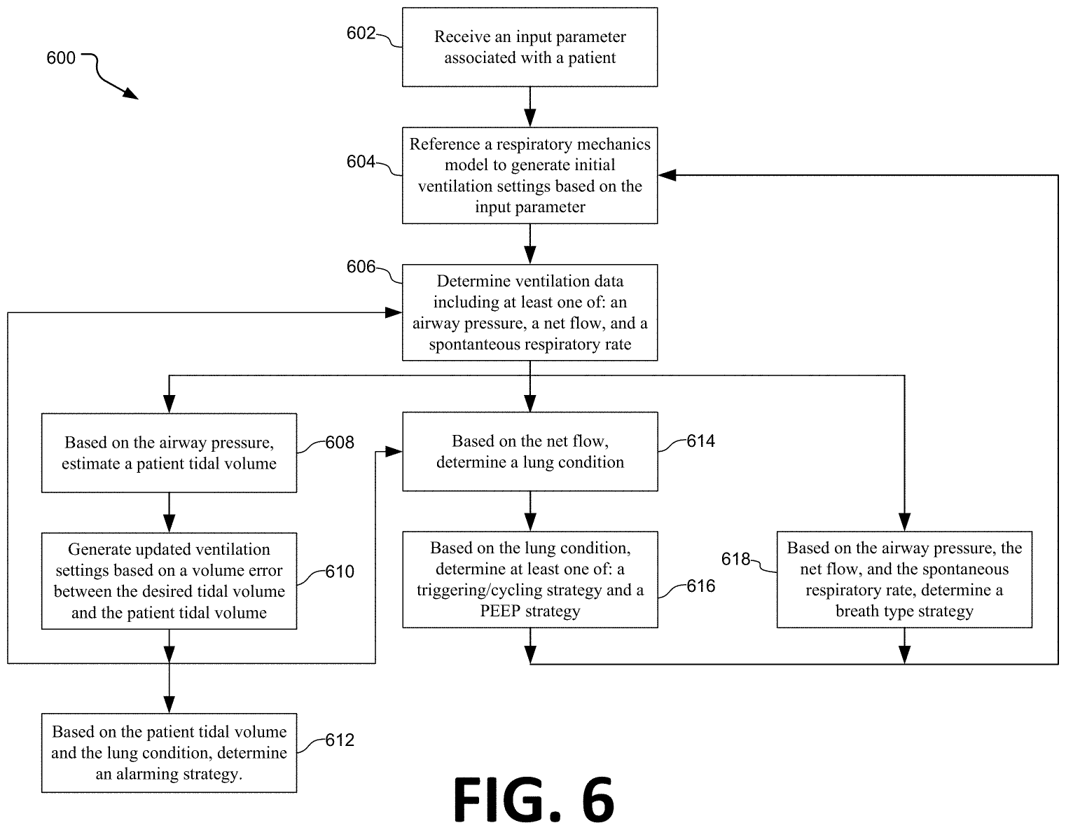

FIG. 6 is a flowchart illustrating a method 600 for one-touch ventilation mode, including ventilation strategies. The method 600 begins at operation 602 where the ventilator that is performing one-touch ventilation mode receives an input parameter associated with a patient. The input parameter is further described herein. Operation 602 may be similar to operation 502 as discussed with respect to FIG. 5.

At operation 604, which may be similar to operation 504 in FIG. 5, the ventilator may reference a respiratory mechanics plane to generate initial ventilation settings, based on the input parameter. As described herein, referencing the respiratory mechanics plane may include mapping the input parameter to ventilation settings on the plane, or applying the input parameter to the plane to generate ventilation settings. The ventilator may deliver ventilation based on the initial ventilation settings. The initial ventilation settings may include one or more of a desired tidal volume, a desired respiratory rate, and/or a desired distending pressure. The ventilator may obtain ventilation data associated with a patient including at least one of: an airway pressure, a net flow, and a spontaneous breathing rate, at operation 606. The ventilation data may be obtained during ventilation of the patient. The desired tidal volume may be an input into a volume-targeted pressure control system, such as volume-targeted pressure control system 412, where an airway pressure is targeted to minimize a volume error between delivered lung volume and desired tidal volume. The airway pressure is adjusted to minimize the volume error and may thus based on desired tidal volume. A net flow may also vary as the ventilation settings are adjusted to target the desired tidal volume. Additionally, a spontaneous breathing rate may be determined based on the desired tidal volume by accounting for the patient's efforts when determining delivered lung volume. The airway pressure, net flow, and spontaneous respiratory rate may be used by the one-touch ventilation mode individually or in combination. As shown in method 600, operation 606 may split into operations 608-610, 608-614, 614-616, and 618. Although these operations may be independent of each other, it should be appreciated that these operations 608-610, 608-614, 614-616, and 618 may occur concurrently, contemporaneously, or simultaneously.

For example, at operation 608, the ventilator may estimate a patient tidal volume (or delivered lung volume) based on the airway pressure. As an example, the ventilator may use a volume-targeted pressure control system to correlate the airway pressure with a delivered lung volume, as further described in FIGS. 4A and 4B. At operation 610, the ventilator may generate updated ventilation settings based on a volume error between the desired tidal volume and the patient tidal volume. For example, these updated ventilation settings may continually update in a closed-loop system, such that operations 606-610 may repeat similar to the operations of the system described in FIG. 4B. As another example, ventilation settings may be updated continually to minimize the volume error and target a delivered lung volume based on the desired tidal volume.

The method 600 may continue to operation 612 where an alarming strategy is determined based on the patient tidal volume and a lung condition, such as the lung condition identified at operation 614, below. Some alarming strategies are discussed with respect to FIG. 4A, such as protective alarms and informative alarms. The alarming strategy may change or update with changes in ventilation settings, desired ventilation parameters, and/or other determined ventilation strategies (such as breath type strategy, triggering/cycling strategy, and PEEP strategy).

As another example, at operation 614, the ventilator may estimate, determine, or identify a lung condition based on the net flow. For example, the ventilator may determine a lung condition based on a lung conditions identification algorithm, such as lung condition identification component 428. Based on the lung condition, the ventilator may determine at least one of: a triggering/cycling strategy and a PEEP strategy, at operation 616. These strategies may be similar to those described with respect to FIG. 4A.

As a further example, at operation 618, the ventilator may determine a breath type strategy based on the airway pressure, the net flow, and the spontaneous respiratory rate. As an example, a breath type strategy may be determined similar to that discussed in FIG. 4A.

The updated ventilation settings generated at operation 610, the triggering/cycling strategy and PEEP strategy determined at operation 616, and the breath type strategy determined at operation 618 may provide feedback data (e.g., breath type feedback data 436 and PEEP strategy feedback data 438) to the NRM plane and/or the RR plane at operation 602. Thus, the method 600 may repeat operations 604-618 as required or desired. For example, the updated ventilation settings generated at operation 610 (to minimize volume error) may change the location of the patient's point on the respiratory mechanic's plane, and/or may change desired ventilation parameters at operation 604. A change in the patient's point and/or the desired ventilation parameters may cause a corresponding change or update to the generated ventilation settings.

Although the present disclosure discusses the implementation of these techniques in the context of a ventilator capable of performing a one-touch ventilation mode, the techniques introduced above may be implemented for a variety of medical devices or devices utilizing flow sensors. A person of skill in the art will understand that the technology described in the context of a medical ventilator for human patients could be adapted for use with other systems such as ventilators for non-human patients or general gas transport systems. Additionally, a person of ordinary skill in the art will understand that the modeled exhalation flow may be implemented in a variety of breathing circuit setups.

Although this disclosure describes referencing a specific set of respiratory mechanics planes (e.g., the NRM plane and the RR plane), it should be appreciated that any other reference plane or model may be used. Additionally, it should be appreciated that the described respiratory planes may be updated or adjusted based on other parameters, or may be customizable based on available input parameters.

Those skilled in the art will recognize that the methods and systems of the present disclosure may be implemented in many manners and as such are not to be limited by the foregoing aspects and examples. In other words, functional elements being performed by a single or multiple components, in various combinations of hardware and software or firmware, and individual functions, can be distributed among software applications at either the client or server level or both. In this regard, any number of the features of the different aspects described herein may be combined into single or multiple aspects, and alternate aspects having fewer than or more than all of the features herein described are possible.

Functionality may also be, in whole or in part, distributed among multiple components, in manners now known or to become known. Thus, a myriad of software/hardware/firmware combinations are possible in achieving the functions, features, interfaces and preferences described herein. Moreover, the scope of the present disclosure covers conventionally known manners for carrying out the described features and functions and interfaces, and those variations and modifications that may be made to the hardware or software firmware components described herein as would be understood by those skilled in the art now and hereafter. In addition, some aspects of the present disclosure are described above with reference to block diagrams and/or operational illustrations of systems and methods according to aspects of this disclosure. The functions, operations, and/or acts noted in the blocks may occur out of the order that is shown in any respective flowchart. For example, two blocks shown in succession may in fact be executed or performed substantially concurrently or in reverse order, depending on the functionality and implementation involved.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurements techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A method for controlling a medical ventilator, the method comprising:
receiving, at the medical ventilator, an input of intrinsic information associated with a patient;
applying the intrinsic information to a respiratory mechanics plane to generate initial ventilation settings;
delivering pressurized ventilation according to the initial ventilation settings and acquiring ventilation data;
applying the acquired ventilation data to the respiratory mechanics plane to generate updated ventilation settings; and
delivering subsequent ventilation according to the updated ventilation settings.

2. The method of claim 1, wherein the respiratory mechanics plane is at least one of: a normalized respiratory mechanics (NRM) plane and a respiratory rate (RR) plane.

3. The method of claim 1, wherein the acquired ventilation data is a compliance of the patient and the updated ventilation settings are associated with a desired distending pressure.

4. The method of claim 1, wherein applying the acquired ventilation data to the respiratory mechanics plane includes determining a patient status point on the respiratory mechanics plane.

5. The method of claim 4, wherein the respiratory mechanics plane includes a preferred region of ventilation, and wherein applying the acquired ventilation data to the respiratory mechanics plane further includes comparing the patient status point and the preferred region of ventilation.

6. The method of claim 5, wherein the intrinsic information is a predicted body weight of the patient.

7. The method of claim 1, wherein the acquired ventilation data is one of: a spontaneous breath rate, an expiratory time constant, PEEP, a patient effort, an airway pressure, a compliance, and an oxygen saturation.

8. The method of claim 7, wherein the acquired ventilation data is associated with a ventilation strategy, wherein the ventilation strategy is at least one of: a breath type strategy, an alarming strategy, a triggering strategy, a cycling strategy, and a PEEP strategy.

9. The method of claim 8, wherein the acquired ventilation data is the expiratory time constant and the ventilation strategy is the PEEP strategy.

10. The method of claim 9, wherein delivering subsequent ventilation includes changing one of: an inhalation flow or an exhalation pressure.

11. A method for controlling a medical ventilator, the method comprising:
receiving an input of intrinsic information associated with a patient;
mapping the intrinsic information to initial ventilation settings on a respiratory mechanics plane, the initial ventilation settings including at least an initial tidal volume setting and an initial pressure setting;
delivering initial ventilation according to the initial ventilation settings;
during initial ventilation, determining a net flow value;
based on the net flow value, determining a lung condition;
based on the lung condition, determining a trigger type and a PEEP protocol; and
delivering subsequent ventilation based on the determined trigger type and the PEEP protocol.

12. The method of claim 11, the method further comprising:
based on the PEEP protocol, increasing a PEEP level.

13. The method of claim 12, the method further comprising:
applying the PEEP protocol to the respiratory mechanics plane to generate updated ventilation settings; and
delivering the updated ventilation settings.

14. The method of claim 11, wherein determining the lung condition comprises:
determining an expiratory time constant of an exhalation phase of the patient; and
comparing the expiratory time constant with a time constant threshold to identify the lung condition.

15. The method of claim 11, wherein the trigger type is one of: a flow trigger type, a pressure trigger type, a signal distortion trigger type, or a synchronized trigger type.

16. A method for controlling a medical ventilator, the method comprising:
initiating positive pressure ventilation with one-touch input, the one-touch input indicating intrinsic information associated with the patient;
mapping the intrinsic information on a respiratory mechanics plane to determine initial ventilation settings;
delivering the positive pressure ventilation according to the initial ventilation settings, without requiring further input from a clinician;
during ventilation of the patient, measuring ventilation data including at least one of: a net flow value, an airway pressure value, or a spontaneous respiratory rate value;
mapping the measured ventilation data on the respiratory mechanics plane to determine updated ventilation settings; and
delivering subsequent positive pressure ventilation according to the updated ventilation settings.

17. The method of claim 16, wherein the initial ventilation settings include at least an initial tidal volume setting and an initial pressure setting.

18. The method of claim 17, wherein the measured ventilation data includes the net flow value, the airway pressure value, and the spontaneous respiratory rate value.

19. The method of claim 17, wherein the measured ventilation data includes a lung condition determined based on the net flow value.

20. The method of claim 16, wherein the measured ventilation data includes a lung condition determined based on the net flow value and a patient tidal volume based on the airway pressure value.
